(19) United States
(12) Patent Application Publication
(10) Pub. No.: US 2021/0350886 A1
(43) Pub. Date: Nov. 11, 2021

(54) Multi-Segment Central Medical Records Storage, Security, And Access

(71) Applicant: AssureCare LLC, Cincinnati, OH (US)

(72) Inventor: Steven Joos, Cincinnati, OH (US)

(21) Appl. No.: 17/314,081

(22) Filed: May 7, 2021

Related U.S. Application Data
(60) Provisional application No. 63/022,705, filed on May 11, 2020.

(51) Int. Cl. G16H 10/60; G06F 21/62; G06F 16/2457

(52) U.S. Cl. CPC G16H 10/60; G06F 16/2457; G06F 21/6245

(57) ABSTRACT

A multi-segment health data system comprises a computerized central shared record repository, security matrix, and operations configuration matrix, whereby the security matrix controls which actors are able to view, use, and manipulated which data in the shared repository record, and the operations configuration matrix allows dynamic changes to the ways in which data is accessed and experienced by various users and operations. The central shared record is shared among plural organizations, such as such care providers, government agencies, private insurers, pharmacies, and prescription benefit plans to provide a secure, complete, and centrally located medical and health record which includes both shared and proprietary data elements to accommodate record permanence and rapid adaptation to shifting data ownership and other changes in access requirements.

### CROSS-REFERENCE TO RELATED APPLICATIONS

This application claims the benefit of U.S. Provisional Patent Application Ser. No. 63/022,705, filed on May 11, 2020, the disclosure of which is hereby incorporated by reference as if set forth in its entirety herein.

### BACKGROUND

The present disclosure pertains to population management information technology involving the handling of medical records and other information useful in advancing the health and well-being of individuals and groups.

### SUMMARY

A multi-segment health data system comprises a computerized central shared record repository, security matrix, and operations configuration matrix. The security matrix controls which actors are able to view, use, and manipulate data in the shared record repository. The operations configuration matrix allows dynamic changes to the ways in which data is accessed and experienced by various users, such as human users and automated compliance and analysis operations.

The multi-segment health data system allows multiple user segments, such care providers, government agencies, private insurers, pharmacies, and prescription benefit plans to share a secure, complete, and centralized medical and health record which includes both shared and proprietary data elements, without comprising the privacy of an individual's data or inadvertent disclosure of proprietary data among multiple tenants who use the system. For example, multiple tenants within a segment, such as multiple pharmacies, may also access a single shared patient record, and also store proprietary information for their own use within that record without, other parties obtaining unpermitted access to proprietary data stored as part of the larger central shared record repository.

The information stored for an individual may include, but is not limited to, a medical record. For example, the repository may include information on non-medical health and well-being programs and activities with which an individual is involved.

The operations configuration matrix allows dynamic adjustment of user work flows, for example, and may also be used to configure background processes such as patient and population analytics. Combining the shared data, security matrix, and operations configuration matrix allows for central, secure access to complete records be implemented in a cost-effective way, with minimal coding, while enjoying separate checks and balances for data integrity and security. Data integrity may be preserved through central accumulation, while shifting data access privileges are accommodated by masking, unmasking, and re masking access to aspects of data as required.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to limitations that solve any or all disadvantages noted in any part of this disclosure.

### BRIEF DESCRIPTION OF THE FIGURES

A more detailed understanding may be had from the following description, given by way of example in conjunction with the accompanying drawings.

FIG. 1 illustrates an example complete central record for an individual.

FIG. 2 illustrates occlusion of certain data elements of the record of FIG. 1 when accessed by a particular user or process, as may be achieved by a security matrix.

FIG. 3 illustrates a graphical user interface (GUI) that may be used to display a portion of an individual's central record.

FIG. 4 illustrates data access segmentation and security and operations configuration matrix controls.

### DETAILED DESCRIPTION

A multi-segment health data system comprises a central shared record repository, a security matrix, and an operations configuration matrix. The security matrix controls which actors are able to view, use, and manipulate data in the shared repository record. The operations configuration matrix allows dynamic changes to the ways in which data is accessed and experienced by various users and operations.

### Example Advantages

Traditionally, data in health care is highly fragmented. Information is divided among health providers, health plans, and other organizations, such as pharmacies and community resources. The main drivers of this division are historical, legal, and competitive. Organic introduction of information technology over the past 25 years, for example, has result in widely varying models for similar functions as each organization implements creates and implements separate business workflows idiosyncratically. Legal and compliance restrictions, along with protecting confidential information and perceived competitive advantages, hamper the willingness of organizations to share data. This fragmentation creates significant overhead costs when data does need to be shared, as prevents leveraging data sets to their full power to improve individual lives and community health outcomes.

There have been several initiatives to bring disparate data together for reference across health care organizations, including Health Information Exchanges (HIE) and the Medicaid Information Technology Architecture Initiative (MITA) from the CMS. MITA especially calls out a roadmap of bringing all government health care beneficiaries to have portable data records across the country to the end state of being “pre-enrolled based on clinical and administrative data across programs and state boundaries.” No current solution in the market supports this unified view of a human life in a single data set, which can be leveraged by multiple health care actors. As a result, the health care organizations miss numerous opportunities to holistically assess risk, proactively manage care, and connect to people to beneficial services.

In contrast, the multi-segment health data system described herein provides data integrity by acting as a central, secure repository, while providing security in the retention of data despites shifts in plans, providers, policies, or other events. Further, the multi-segment health data system can provide confidential analytics of the broader data set either for the benefit for an individual or of a population. For example, without alerting a provider to confidential information available from other sources, the system may be configured to recommend a course of action, such as a screening test or examination, based on information drawn from a variety of sources. Similarly, the system may be able to inform a health care policy for a provider or payer, for example, based on healthcare outcomes for select classes of individuals with a given set of indications and treatment outcomes.

The matrixing of security and operations configuration provides efficient implementation of complex security across different segments which, while having a shared need for central and complete records, have different legal standing regarding their access to such information, as well as highly varied work flows. Similarly, the matrixing of security and operations configuration allows multiple tenants, e.g., multiple legal entities within a single user segment, to obtain highly tailored access. Direct competitors, for instance, may share the advantages of a centralize record repository. The use of a central repository has a number of advantages. For example. by using a common, secure, central medical record, potential issues of incomplete or inaccurate records are avoided. At the same time, security matrixing provides integrity of access policies. The combination allows the storage of private data centrally, where it may be guarded and accessed for important public purposes, while nonetheless guarding proprietary interests of various parties interested in access to the shared information.

Improvements in health outcomes are expected from enabling a wide-variety of audiences to holistically manage a human life. Improvements in cost are expected from driving down the total cost of data ownership by isolating discrete configuration sets allowing for quicker deployments without having to retest configuration from other segments.

Improvements in data integrity and data quality are also expected. Data integrity is improved through reducing transferring data between entities who require access to a shared dataset. By restricting select data access through a segment ID and possibly down to specific legal flags date ranges, different business entities can view common information to a patient, while still being able to preserve compliance and contractual data segregation.

Improvements are expected in expanded preventative analytics. The multi-segment solution described herein allows predictive algorithms to run on content that is relevant but should not be made fully available to certain actors within the system. For example, a person with a certain insurance that leverages two pharmacy chains for their prescriptions would have a complete view to identify adherence issues, without exposing the restricted information to the individual pharmacy chains.

Combining the use of an operations configuration matrix with a security matrix and shared central repository record has operation advantages. In complex implementations, varied workflows are configured to the needs of specific business units. Matrixing this configuration allows lower total cost of ownership and maintenance among parties sharing similar needs. For example, by allowing updates by that are targeted by user segment, unnecessary testing of unaffected segments may be avoided. This is especially helpful in Medicaid programs and other situations where changes laws and standards occur frequently.

The multi-segment approach provides an opportunity for a health care organization to maintain a single record of care for a human life along with the necessary controls to manage the regulatory and competitive concerns of sharing that data among different actors. It combines the advantages of a multi-tenant approach, whereby a deployment may include multiple configurations and views into for a person's data, with the ability to share a single record per individual member/patient across user segments. In contrast, traditional multi-tenant approaches require one unique record per person within each organization. Data quality, and data insights, suffer dramatically from data fragmentation among health care organizations, even when they use the same software multi-tenant software on their separate data repositories.

Shared Data Record

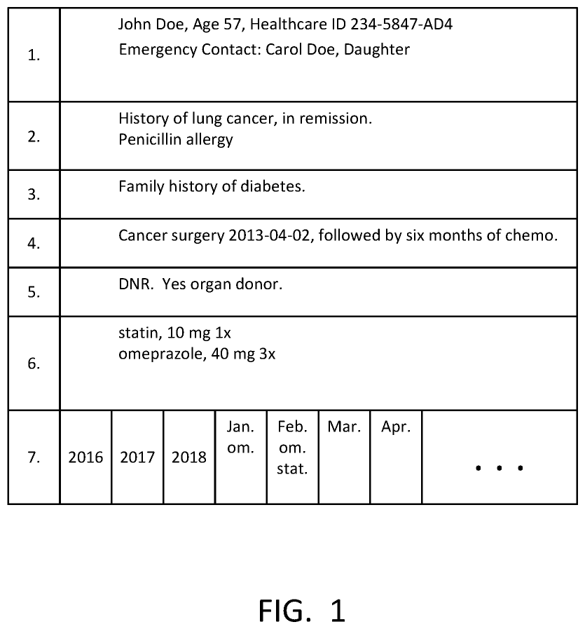

FIG. 1 illustrates an example portion of a shared electronic medical record for an individual in a shared record repository of a multi-segment health data system. The record of the example of FIG. 1 is divided into: (1) personal identification information; (2) personal medical history; (3) family medical history; (4) treatment history; (5) medical directives; (6) prescriptions; and (7) prescription filling records. The personal identification information (1) may include, for example, name, address, age, medical plan ID numbers, etc. The personal medical history (2) may include allergies, prior diagnoses and treatments, etc. The family medical record (3) may include things like serious conditions that have affected family members, such as cancer and heart disease. The treatment history (4) may include former pharmaceutical and surgical treatments and results. The medical directives (5) may include, e.g., living will indications of the patient's wishes in case the patient is unable or unavailable to speak for the themselves. The prescriptions (6) may be current medications, and the prescription filling records (7) may reflect recent filling of prescriptions by one or more pharmacies. In the example of FIG. 1, all of this data is held in a single system, e.g., as part of a larger database of medical information.

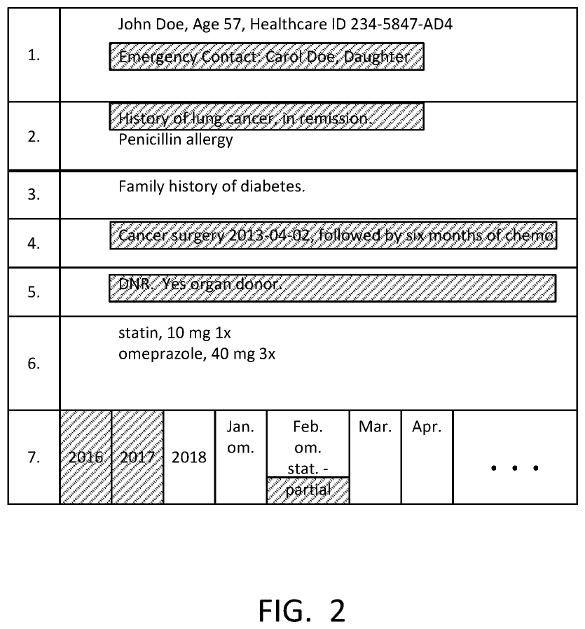

FIG. 2 shows the shared electronic medical record of FIG. 1 overlaid by a security filter of a security matrix for a tenant of a multi-segment data system. In the example of FIG. 2, the tenant is a pharmacy, ABC Drugs, in April of 2019. The pharmacy is able to view certain aspects of the shared electronic medical data record, but not all. For example, the pharmacist will require sufficient personal information to confirm the identity of the person for whom a prescription is being filled, and may further rather information regarding medication allergies or prior reactions. However, a pharmacist is unlikely to need access to medical directives or prior conditions which are not pertinent to current diagnoses or drug indications. Therefore, the pharmacist is blocked by the security matrix from viewing certain portions of the record.

The security filter also controls which data the user may add, delete, or modify. In the example of FIG. 2, ABC Drug is able to add data to the record. The added data may be viewable by other tenants, as required. For example, when ABC Drug fills a prescription, this fact is recorded centrally. The added data may include information which is proprietary to ABC Drug and not viewable by other parties. For example, the record may include a note that a prescription was only partially filled. Notably, ABC Drug may store this information in the central shared electronic medical record, rather than needing to store this is a separate proprietary system.

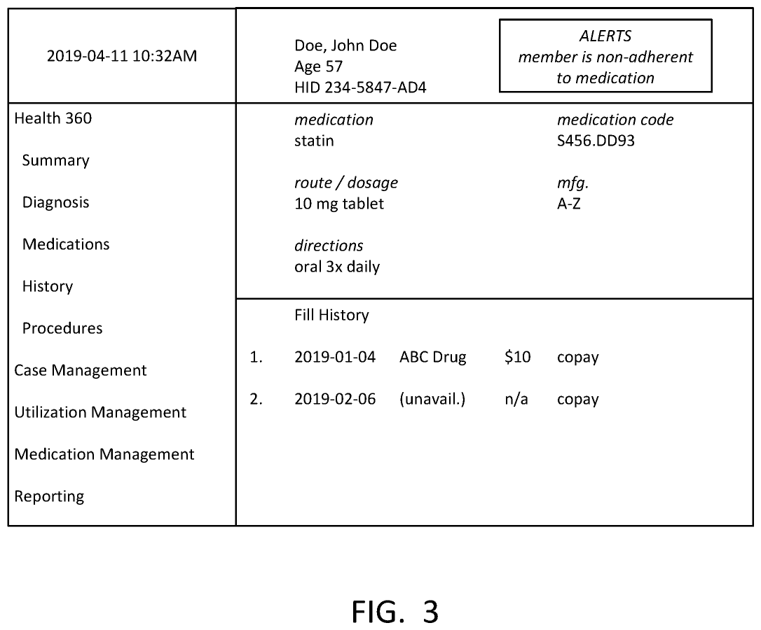

In the example of FIG. 3, a pharmacist at ABC drug will be allowed, by the security matrix, to see some of the patient's person information, but not all of it. Similarly, the pharmacist may see relevant medical history, such as a current allergy, but not the complete medical record. The pharmacist can see the current valid prescriptions. However, in this example, the pharmacist is not permitted to view the complete prescription fill record. For example, a private insurer may have only authorized the pharmacy as an approved vendor for reimbursement as of 2018. The pharmacy may be blocked from seeing older prescription fills older than 2018. Similarly, not shown, the entire record may be divided by date, with access for particular segments or tenants limited by date.

In the example of FIG. 3, the pharmacist will be able to see that the prescription for the omeprazole was filled in January and March, but not in March, and so far not in April. The pharmacist will also be able to see that the statin was filled in February, but not in January or March, and so far not in April. Therefore, the pharmacist may fill the prescriptions in April, and add data to that effect for April.

However, in this example, the pharmacist cannot see that the filling of the statin script in February was a partial filling. For example, the filling of the statin script in February may have been done by another pharmacy that has not agreed to share such data with ABC Drug. In the example, the partial filling information is kept by the multi-segment system as proprietary to the other pharmacy.

Alternatively, using the multi-segment system, pharmacies may easily share such information via modification of the central record, establishment of appropriate security filtering, and configuration of work flows and data access. Once the proprietary data details are captured centrally, there is great flexibility to later adjust access without the logistical challenges of trying to gather such information from disparate systems. The treatment of the data may be altered retroactively. For example, if ABC Drug and the other pharmacy are merged, a new data system is not required. Rather, the security filter and access configuration can be adjusted in the existing multi-segment system, with no need for new software or data migration per se.

FIG. 3 is a screen shot of an example user interface for a tenant to view the example shared electronic medical record of FIG. 1. The data which a tenant may view and manipulate is regulated by the security matrix. A separate configuration matrix controls the way in which the data is viewed or manipulated. This helps to ensure the integrity of security aspects, regardless of changes to work flows and user interfaces, for example, while permitting the maximum flexibility and reuse of work flow and user interface development efforts.

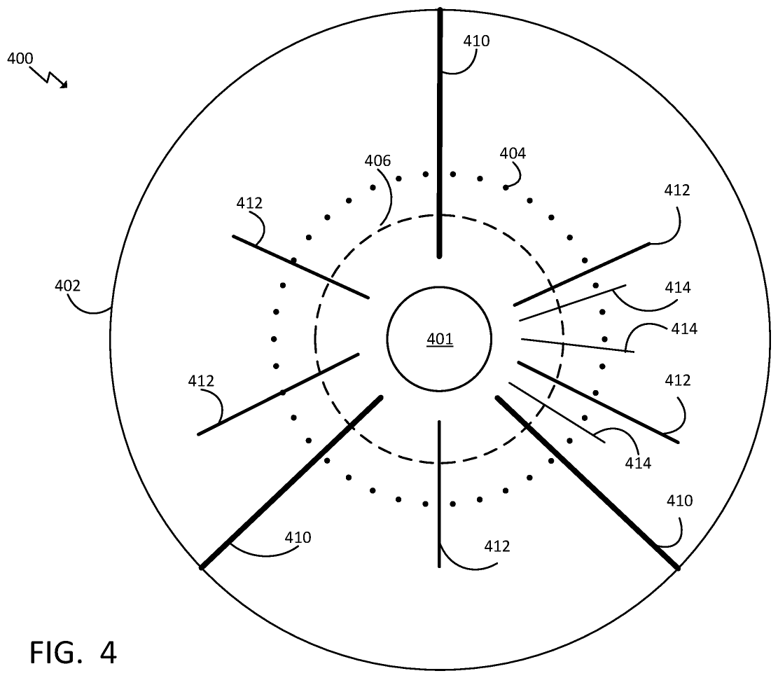

FIG. 4 illustrates data divisions in a multi-segment health data system 400. The system includes central shared record repository 401, a security matrix 406, and an operations configuration matrix 404. The security matrix 404 controls which actors in the whole set of users 402 are able to view, use, add, delete, and manipulate data in the shared repository record 401. The operations configuration matrix 404 allows dynamic changes to the ways in which data is accessed and experienced by various users and operations. The security matrix 406 controls which users are able to access which aspects of the central shared record repository 401.

Data access is divided by segment boundaries 410. For example, aspects of the security matrix 406 and operations configuration matrix 404 may be common to all users within a segment. For example, the different user segments may be care providers, wellness services, insurers, or state agencies.

In the example of FIG. 4, each segment is divided by tenant boundaries 412, where certain security or configuration aspects of data access may be common within a tenant, but differentiated by tenant within a segment. For example, different tenants may be competing care providers or pharmacies. Further, data access may be refined by user boundaries 414, which divide the security matrix 406, and operations configuration matrix 404 at a user class or individual user level, for example.

For simplicity, FIG. 4 illustrates segmentation of users, but not the segmentation of individuals whose records reside in the central shared repository 401. Within a multi-segment health data system, a segment may also refer to a segment of a population of patient/member individuals that is identified by, for example, a certain set of care or accounting needs, or historical data classification. Viewing segmentation in this way is advantageous for coordinating operations configuration, for example, because different segments may have different historical data structures or reporting needs. Although not shown in FIG. 4, it will be appreciated that the security matrix 406 and the operations configuration matrix 404 may equally accommodate segmentation of users and segmentation of individuals.

Referring again to FIG. 4, the central shared record repository 401 contains a comparatively complete member/patient record, including both current and past records of medical and other wellness care. For example, historical data in an individual's record may include retrospective service authorization where a member's coverage has changed between two benefit years, and the service rendered happened under the prior benefit. The record is not limited to the care provided under the present care provider or insurer, for instance.

The operations configuration matrix 404 and security matrix 406 manage what they can view and act upon. The operations configuration matrix 404 may be tailored both from a member perspective and a user perspective. In other words, there may be variations by individual and segments of individuals, as well as by users, tenants, and segments of tenants and users. For example, there may be variations not only within organizations but within different population groups for things like health assessments, service authorization types, medication management activities, or views into data elements that do not apply to another segment.

The multi-segment allows the specific user access to change both by record and the specific interaction, e.g., as opposed to being coded based merely on a user's functional role. The segmentation of the user, e.g., a patient's association, as well as the context, e.g., the clinical, compliance, or research nature of the interaction, also factor into the functioning of the security matrix 406 and the operations configuration matrix 404.

Further, access may be fine-tuned down to controlling the section or even field level to manage access to data, e.g., according to legal flag or specific date range. Configurations can be adjusted and deployed by user or patient segment, and deployed independently of other segments to eliminate having to test adjacent segments. Configurations may be shared and inherited among segments.

The use of a central shared record repository, in combination with the security and configuration matrixes, provides the opportunity for unusual visibility into the care of individuals, segments of populations, and whole populations. For example, it may be possible to view on a single user interface display screen: service authorization types and specific requirements; turnaround times on requests for service; assessments, especially health risk assessments (which vary by state); benefits available by date; reporting requirements; and/or contact management rules and processes.

We claim:

1. A method of using a system, the system comprising:
a data repository, the data repository being a computerized multi-segment health data central shared record repository comprising shared and proprietary data elements, the data repository pertaining to medical records of individuals, the individuals belonging to patient segments of a population;
a security matrix, the security matrix comprising settings for access to the medical records of individuals in accordance with one or more segments of the population with which each individual is associated, the security matrix further comprising settings for access to the medical records of individuals in accordance with one or more segments of users; and
an operations configuration matrix comprising instructions for the manipulation of data of the data repository in accordance with one or more segments of the population with which each individual is associated and one or more segments of users with which a user is associated,
the method comprising:
providing an interface to the user in accordance with the security matrix and the operations configuration;
querying the data repository in accordance with a user criterion;
masking, at a first time and in accordance with the security matrix, a portion of a result of the query.

2. The method of claim 1, wherein the user is a human user and the interface is a graphical user interface.

3. The method of claim 1, wherein the user is an automated compliance process and the interface is a computer connection.

4. The method of claim 1, wherein the portion of the result of the query is proprietary to a first user segment and the user input is received by a user in a second user segment.

5. The method of claim 4, wherein the method further comprises:
at a second time after the first time, altering the security matrix to allow users of the second user segment to have access to the proprietary data of the first user segment.

6. The method of claim 5, wherein the method further comprises:
at a third time after the first time and the second time, altering the security matrix to again mask users of the second user segment from having access to the proprietary data of the first user segment.
